(19) United States
(12) Patent Application Publication
(10) Pub. No.: US 2012/0015022 A1
(43) Pub. Date: Jan. 19, 2012

(54) Biodegradable wound care products with biocompatible artificial skin treatment

(75) Inventor: Kenneth P. Glynn, Flemington, NJ (US)

(73) Assignee: Speechswitch, Inc.

(21) Appl. No.: 12/803,992

(22) Filed: Jul. 13, 2010

(51) Int. Cl. A61K 9/70; A61K 36/03; A61P 17/02; A61K 36/02

(52) U.S. Cl. 424/447; 424/195.17

(57) ABSTRACT

A biodegradable wound care product with biocompatible artificial skin treatment is described. It includes a biodegradable bandage support member and a biodegradable adhesive on at least a portion thereof. The support member is made of a biodegradable material, such as natural or artificial textiles, plastics or papers. The biodegradable actives carrier is attached to a portion of the biodegradable bandage support member and is a synthetic or natural biodegradable adhesive or a combination thereof. The active ingredient located on the biodegradable actives carrier includes a layer of biocompatible algae artificial skin including sterilized water and at least sixty percent by weight of reconstituted seaweed. Laminaria seaweed may be used for its desirable polyfucose sulfate content. The active ingredient is prepared by first drying the raw materials, converting the raw materials to a powder and then reconstituting the powder with sterilizes water and other possible actives and/or inerts.

### BACKGROUND OF INVENTION

a. Field of Invention

The invention relates generally to biodegradable wound care products with biocompatible artificial skin treatments. More specifically, the present invention relates to bandages comprised of a biodegradable support member, a biodegradable adhesive, a biodegradable actives carrier and an active ingredient. The active ingredient is intended to assist in the healing process of wounds and includes a layer of biocompatible algae artificial skin including sterilized water and at least sixty percent by weight of reconstituted seaweed. Brown Laminaria seaweed may be used for its desirable polyfucose sulfate content. The active ingredient is prepared by first drying the raw materials, converting the raw materials to a powder and then reconstituting the powder with sterilizes water.

b. Description of Related Art

The following patents are representative of the field pertaining to the present invention:

U.S. Pat. No. 5,100,879 to Ueno et al. describes a method of disinfection for retroviruses which comprises contacting inanimate object in need of such treatment with a natural or synthetic oligo- or polysaccharide having at least one S-oxoacid group attached to the saccharic carbon atom through a linking group of low molecular weight.

U.S. Pat. No. 5,232,769 to Yamoto et al. describes a microcapsule having a particle diameter of 2.about.300 .mu.m and comprising a substance acting to improve physiological conditions of human skin, for example, substances exhibiting such effects as skin whitening, aging preventive, humidity preservable, itch suppressive, pain-killing, or antiphlogistic ones, and/or aromatic agents contained within the filmy coating of synthetic high molecular substance. The microcapsule is not broken when making, processing, or laundering the textile structure, but is gradually broken when the textile structure is put on the human body, used for another purpose, or subjected to intentional application of friction or pressure thereto, and sustainedly releases acting substances contained therein. Treatment liquids comprising these microcapsules and binder, preferably containing a spraying agent, adapt the microcapsules to tightly adhere to textile structures such as stockings underwear, and bedclothes, thereby providing a textile structure to exhibit the aforesaid effects.

U.S. Pat. No. 5,487,889 to Eckert et al. describes a biological bandage, comprising an envelope enclosing cells that secrete biologically active cellular products such as growth factors, which promote the healing of wounds. The envelope is further comprised of a permeable bottom membrane through which the cellular product diffuses, and a top membrane. Preferably the bandage has a separator interposed between the two membranes. This invention also relates to a method for treating wounds. The bandage provides a continuous, uniform source of fresh cellular product.

U.S. Pat. No. 5,575,995 to Giovanoni describes a method of treating lesions surgically or otherwise induced on living tissue. Lesions surgically or otherwise induced on living tissue are treated by applying to the tissue a gel containing a water solution of ferric subsulfate, glycerin to thicken the solution and polyvinyl pyrrolidone as a film forming material.

U.S. Pat. No. 5,836,970 to Pandit describes a wound dressings comprising a synergistic combination of reagents comprising a blend or mixture having effective amounts of chitosan and alginate to provide an absorption capacity for wound exudate far greater than that obtainable with the same amount by weight of either chitosan or alginate alone, along with the inherent hemostatic properties of the alginate and the other biological properties of the reagents including, but not limited to fungistic and bacteriostatic properties as well as the ability to accelerate wound healing by inducing high migration of poly- and mononuclear cells toward the wound and rapid granulating tissue formation with abundant angiogenesis. The alginate and chitosan may be provided in the form of a powder, film, gel, foam or mixtures thereof. In addition to the alginate and chitosan, the wound dressing may and frequently will contain other reagents providing specific desired functions. The dressing may be applied directly to the wound surface or carried on a suitable substrate applied to cover the wound.

U.S. Pat. No. 6,211,296 B1 to Frate et al. describes a blend of one or more crosslinked polymers or copolymers prepared from an olefinically unsaturated polymerizable carboxylic or anhydride monomer and at least one high molecular weight essentially linear polymer prepared from an olefinically unsaturated polymerizable carboxylic acid monomer. The blend is neutralized to typically a low pH, and generally contains a noncovalent, e.g. ionic, crosslinking agent, and a cure rate modifier. The hydrogel blend is capable of containing a large amount of water, has good tack, good adhesion, and can be utilized as an application vehicle having a backing thereon as well as containing various substances such as personal care compounds, pharmaceuticals, active ingredients, and the like.

U.S. Pat. No. 6,419,962 B1 to Yokoyama et al. describes an external skin treatment composition that comprises a “unicellularized” plant. This composition exhibits superior stability of the effective plant ingredients, superior moisture retention, and superior elimination of specific free radicals, and it offers sustained release of the effective plant ingredients. Methods are disclosed to unicellularize plants with, for example, an enzyme.

U.S. Pat. No. 6,627,215 B1 to Dale et al. describes devices and compositions for the management of infection of topical lesions, each of the devices and compositions containing protonated/acidified nucleic acids either on its surface, or integrated into the device. These modified nucleic acids are effective as bactericidal and/or bacteriostatic agents without regard to the class of bacteria, so are especially useful when diagnosis is difficult or when multiple infectious organisms are present. The antibiotic activity of nucleic acids of the invention is not dependent on either the specific sequence of the nucleic acid or the length of the nucleic acid molecule. The nucleic acids used in the invention are protonated/acidified to give a pH when dissolved in water of less than pH 7 to about 1, more preferably less than pH 4.5 to about 1, and even more preferably less than pH 2 to about 1.

U.S. Pat. No. 6,723,781 B1 to Frate et al. describes a blend of one or more crosslinked polymers or copolymers prepared from an olefinically unsaturated polymerizable carboxylic or anhydride monomer and at least one high molecular weight essentially linear polymer prepared from an olefinically unsaturated polymerizable carboxylic acid monomer. The blend is neutralized to typically a low pH, and generally contains a noncovalent, e.g. ionic, crosslinking agent, and a cure rate modifier. The hydrogel blend is capable of containing a large amount of water, has good tack, good adhesion, and can be utilized as an application vehicle having a backing thereon as well as containing various substances such as personal care compounds, pharmaceuticals, active ingredients, and the like. Conversely, the hydrogel blend can be dried and subsequently wetted without substantial loss of properties.

U.S. Pat. No. 6,809,231 B2 to Edwards describes a wound dressing containing cellulose-containing material and alginate, wherein the alginate is crosslinked through a polycarboxylic acid ester bond to the cellulose of the cellulose-containing material. A method for preparing a wound dressing is disclosed wherein alginate is crosslinked to cellulose through a polycarboxylic acid ester bond, comprising introducing cellulose-containing material into an aqueous solution wherein the aqueous solution contains water, alginate, a crosslinker, optionally an acid catalyst, and optionally polyethylene glycol to form cellulose-containing material wherein alginate is crosslinked to cellulose through a polycarboxylic acid ester bond, drying, and curing. A wound dressing wherein the wound dressing is prepared by the above method is also described.

U.S. Pat. No. RE 38,827 E to Barrows et al. describes an adhesive composition that may be used to bond or seal tissue in vivo. The adhesive composition is readily formed from a two component mixture which includes a first part of a protein, preferably a serum albumin protein, in an aqueous buffer having a pH in the range of about 8.0-11.0 and a second part of a water-compatible or water-soluble bifunctional crosslinking agent. When the two parts of the mixture are combined, the mixture is initially a liquid which cures in vivo on the surface of tissue in less than about one minute to give a strong, flexible, pliant substantive composition which bonds to the tissue and is absorbed in about four to sixty days. The adhesive composition may be used either to bond tissue, to seal tissue or to prevent tissue adhesions caused by surgery.

U.S. Pat. No. 6,967,261 B1 to Soerens et al. describes a bandage of the type used on acute wounds, minor wounds, burn wounds and irritations, includes a first layer for covering the wound site and an area around the wound site, with the first layer including a top surface and bottom surface; a second layer over the first layer bottom surface, for absorbing exudates from the wound site; the second layer including a poly(ethyleneoxide)-based compound and a chitosan-based compound. A third layer is situated over the second layer, the third layer being of a perforated film, and wherein, at least one antimicrobial agent is associated with the bandage in a position where the antimicrobial agent will come in contact with the wound site, and which is transferable from the bandage to the wound site, upon contact with the wound site.

U.S. Pat. No. 6,974,805 B2 to Hu describes a stable glycosaminoglycan (GSG) structures and methods of use of such GAG structures. These structures comprise a core of free GAG, a coating of crosslinked GAG surrounding the core, and a layer of a positively charged moiety surrounding the crosslinked GAG layer. These GAG structures provide improved stability, both in in vivo and external use. Furthermore, resurfacing of the structures provides improved cell adhesion and thus improved delivery of the GAG into living cells and tissues.

U.S. Pat. No. 7,184,963 B1 to Shannon et al. describes methods for consistently designating appropriate treatment protocols for patients, particularly protocols involving wounds or wound prevention. Specifically, the invention relates to methods wherein a patient condition such as a wound is assessed against defined scales for classifying and grading, which assessment is used in a visual decision tree device to identify one or more components of a treatment protocol.

U.S. Pat. No. 7,361,241 B2 to Barth et al. describes a nonwoven fabric having elastomeric properties spun filaments, consisting preferably (but not essentially) of cellulose material such as cellulose acetate or solvent spun rayon, not in yarn formats, are corrugated or crimped in an overfeed process, as in stuffer box, into stabilized three dimensional batts. A proportion of filaments of a thermal memory material, such as modified polyester, in a stretched format are included. The resulting batt is then subjected to controlled hydroentangling and a controlled heat treatment to yield three-dimensional nonwoven fabrics with elastomeric properties due to contraction of the stretched filaments. Elastomeric memory material activated to shrink by ultrasonic treatment may be used in place of thermal memory material. The elastomeric properties can be adjusted to suit end-use requirements and applications are envisaged in the medical and hygiene areas.

U.S. Pat. No. 7,572,769 B2 to Rapp et al. describes a flowable fibrin adhesive granulate containing thrombin, Factor XIII, fibrinogen, and a calcium salt in the form of granules with a particle size of more than 50 .mu.m to 1000 .mu.m. Said granulate is useful for the healing of wounds in surgery, tissue therapy and/or as supporting material for biological factors. The invention also relates to an effervescent granulate and an effervescent powder for producing a foam that is suitable for hemostasis. The invention further relates to preparations to arrest bleeding containing a non-woven fabric for wounds consisting of a biodegradable supporting material that is coated with a fibrin glue granulate mixture or mixed granulate.

U.S. Pat. No. 7,576,051 B2 to Kurokawa et al. describes a wound dressing capable of accelerating epidermal regeneration. The wound dressing for accelerating epidermal regeneration comprises a polypeptide having at least one species of epidermal regeneration-accelerating minimal amino acid sequences selected from the group consisting of the Arg Gly Asp sequence, the Ile Lys Val Ala Val sequence, and the Tyr Ile Gly Ser Arg sequence and an auxiliary amino acid sequence, a polyalkylenepolyamine and/or polyarylenepolyamine having a weight average molecular weight of 2,000 to 60,000, and a sheet.

U.S. Pat. No. 7,615,236 B1 to Smith describes a medical method and dressing for application, and maintenance of medication on healthy, damaged diseased or infected living tissue. Medication is applied to body tissue and then coated with a bioadhesive providing medication maintenance on tissue and protection from body and other liquids or abrasion thereby preventing removal of the medication during a healing or treatment process.

U.S. Pat. No. 7,655,288 B2 to Bauman et al. describes an implantable bioabsorbable non-woven self-cohered web materials having a high degree of porosity. The web materials are very supple and soft, while exhibiting proportionally increased mechanical strength in one or more directions. The web materials often possess a high degree of loft. The web materials can be formed into a variety of shapes and forms suitable for use as implantable medical devices or components thereof.

Notwithstanding the prior art, the present invention is neither taught nor rendered obvious thereby.

### SUMMARY OF INVENTION

The present invention is directed to a biodegradable wound care product with biocompatible artificial skin treatment. It includes a biodegradable bandage support member and a biodegradable adhesive on at least a portion thereof, a biodegradable actives carrier attached to a portion of the biodegradable bandage support member and an active ingredient located on the biodegradable actives carrier. The active ingredient includes a layer of biocompatible algae artificial skin. The biocompatible algae artificial skin includes sterilized water and at least sixty percent by weight of reconstituted.

In some preferred embodiments of the present invention biodegradable wound care product with biocompatible artificial skin treatment the biodegradable bandage support member is a biodegradable bandage material selected from the group consisting of biodegradable natural fiber textiles, biodegradable synthetic fiber textiles, biodegradable papers, biodegradable plastics and combinations thereof.

In some preferred embodiments of the present invention biodegradable wound care product with biocompatible artificial skin treatment at least one of the biodegradable bandage support members is a cellulosic material.

In some preferred embodiments of the present invention biodegradable wound care product with biocompatible artificial skin treatment the biodegradable adhesive is selected from the group consisting of biodegradable synthetic adhesive, biodegradable natural adhesive, and combinations thereof.

In some preferred embodiments of the present invention biodegradable wound care product with biocompatible artificial skin treatment of the biodegradable adhesive contains a starch-based adhesive.

In some preferred embodiments of the present invention biodegradable wound care product with biocompatible artificial skin treatment the active ingredient is a paste derived from algae that has been dried, converted to a powder, and reconstituted with sterilized water.

In some preferred embodiments of the present invention biodegradable wound care product with biocompatible artificial skin treatment the biodegradable actives carrier is selected from the group consisting of biodegradable natural fiber textiles, biodegradable synthetic fiber textiles, biodegradable papers, biodegradable plastics and combinations thereof.

In some preferred embodiments of the present invention biodegradable wound care product with biocompatible artificial skin treatment the bandage support member has a shape. The shape is selected from the group consisting of circular, rectangular, square, irregular and combinations thereof.

In some preferred embodiments of the present invention biodegradable wound care product with biocompatible artificial skin treatment of further comprises a decorative design on said bandage support member.

In some preferred embodiments of the present invention biodegradable wound care product with biocompatible artificial skin treatment of the bandage support member has a predetermined surface area. The surface area is determined by the desired application.

In some other preferred embodiments, the present invention biodegradable wound care product with biocompatible artificial skin treatment includes a biodegradable bandage support member and a biodegradable adhesive on at least a portion thereof, a biodegradable actives carrier attached to a portion of the bandage support member and an active ingredient located on said biodegradable actives carrier. The biodegradable active ingredient includes a layer of biocompatible algae artificial skin including sterilized water and at least sixty percent by weight of reconstituted Laminaria seaweed.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a bottom view of an embodiment of the present invention biodegradable wound care product with biocompatible artificial skin treatment;

FIG. 2 is a top view of the embodiment of the present invention from FIG. 1;

FIG. 3 is a bottom view of another embodiment of the present invention biodegradable wound care product with biocompatible artificial skin treatment;

FIG. 4 is a flowchart of one production method for the present invention biodegradable wound care product with biocompatible artificial skin treatment; and,

FIG. 5 is a flowchart of another production method for the present invention biodegradable wound care product with biocompatible artificial skin treatment.

### DETAILED DESCRIPTION OF THE EMBODIMENTS

Referring now in detail to the drawings wherein like reference numerals designate corresponding parts throughout the several views, various embodiments of the present invention are shown.

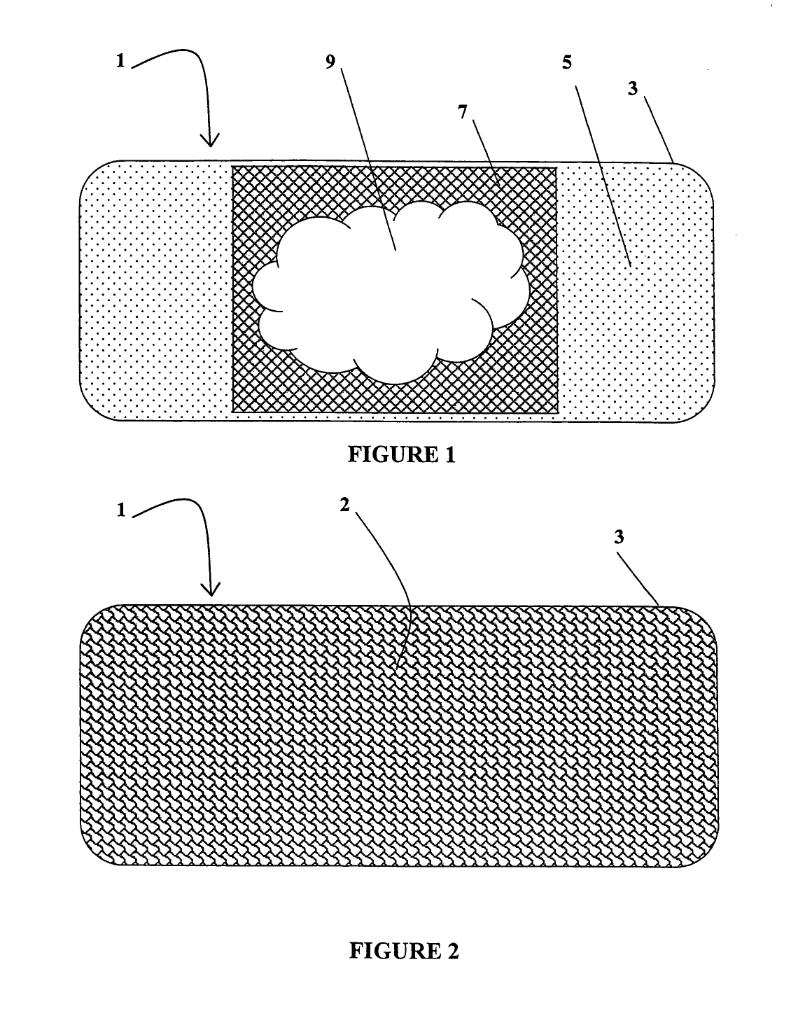

FIG. 1 is a bottom view of an embodiment of the present invention biodegradable wound care product with biocompatible artificial skin treatment and FIG. 2 is atop view of the embodiment of the present invention from FIG. 1. These two Figures are discussed collectively with the understanding that not all reference numerals are in all Figures.

The bottom of present invention biodegradable wound care product with biocompatible artificial skin treatment 1 is shown in FIG. 1. Present invention biodegradable wound care product with biocompatible artificial skin treatment 1 includes a biodegradable bandage support member 3, a biodegradable adhesive 5, a biodegradable actives carrier 7 and an active ingredient 9. In use, the present invention is intended to improve the healing of wounds through the use of active ingredients 9 while offering a product with minimal environmental impact through the exclusive use of biodegradable materials.

The biodegradable bandage support member 3 defines the shape of the present invention biodegradable wound care product with biocompatible artificial skin treatment 1 and provides backing for the present invention. The shape of the biodegradable bandage support member 3 may be selected from the group consisting of circular, rectangular, square, irregular and combinations thereof. The biodegradable bandage support member 3 is constructed out of a biodegradable material selected from the group consisting of biodegradable natural fiber textiles, biodegradable synthetic fiber textiles, biodegradable papers, biodegradable plastics and combinations thereof. In a preferred embodiment of the present invention, a cellulosic material is used for the biodegradable bandage support member 3 due to the desirable material characteristics inherent in cellulosic materials such as low environmental impact and ease of biodegrading.

The biodegradable adhesive 5 is located on a portion of the present invention biodegradable wound care product with biocompatible artificial skin treatment 1. This biodegradable adhesive 5 is adapted to secure the present invention biodegradable wound care product with biocompatible artificial skin treatment 1 to a user while still being biodegradable for disposal after use. The biodegradable adhesive 5 may be selected from the group consisting of biodegradable synthetic adhesives, biodegradable natural adhesives, and combinations thereof. In a preferred embodiment of the present invention, the biodegradable adhesive 5 includes a starch-based adhesive. Starch-based adhesives are readily available and biodegradable, making them an ideal adhesive for the present invention.

Biodegradable actives carrier 7 is attached to a portion of the biodegradable bandage support member 3. In the embodiment of the present invention shown in FIG. 1, the biodegradable actives carrier 7 may be constructed out of a material selected from the group consisting of biodegradable natural fiber textiles, biodegradable synthetic fiber textiles, biodegradable papers, biodegradable plastics and combinations thereof.

The biodegradable actives carrier 7 is adapted to hold an active ingredient 9 and release active ingredient 9 in a controlled manner. The active ingredient 9 is a paste derived from algae that has been dried, converted to a powder, and reconstituted with sterilized water. At least sixty percent by weight of the active ingredient 9 is reconstituted seaweed. Other actives or inerts may be added to the active ingredient 9 to achieve improved wound healing characteristics or other desired traits. In some preferred embodiments of the present invention biodegradable wound care product with biocompatible artificial skin treatment 1 the algae used is Laminaria seaweed. Laminaria seaweeds are desirable due to their high concentrations of poly-fucose sulfate, a fucodin that has been shown to accelerate wound-healing rates.

FIG. 2 shows the top of the embodiment of the present invention biodegradable wound care product with biocompatible artificial skin treatment 1 from FIG. 1. From the top perspective only the bandage support member 3 is visible. A design is printed on the top 2 of bandage support member 3. In FIG. 2 a fish scale design is illustrated on the top 2 of bandage support member 3. It is to be understood that this fish scale design is used as an example and any desired design could be used on the top 2 of bandage support member 3.

The present invention biodegradable wound care product with biocompatible artificial skin treatment 1 has a predetermined surface area that is dependent on the intended application. Individual embodiments should be sized appropriately for their use, such as smaller surface areas for small cuts all the way up to large bandages for burns or surgical applications. The desired application will also determine the shape of the present invention.

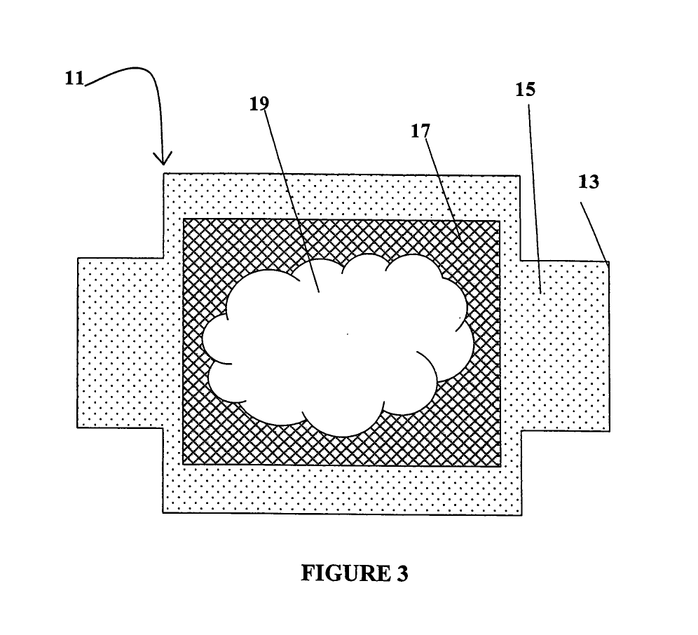

FIG. 3 shows a bottom view of an embodiment of present invention biodegradable wound care product with biocompatible artificial skin treatment 11. Biodegradable wound care product with biocompatible artificial skin treatment 11 is formed as described above in FIG. 1 and includes a biodegradable bandage support member 13, a biodegradable adhesive 15, a biodegradable actives carrier 17 and an active ingredient 19. The biodegradable bandage support member 13 defines the shape of the present invention biodegradable wound care product with biocompatible artificial skin treatment 11 and provides backing for the present invention. The biodegradable bandage support member 13 is constructed out of a biodegradable material selected from the group consisting of biodegradable natural fiber textiles, biodegradable synthetic fiber textiles, biodegradable papers, biodegradable plastics and combinations thereof. In a preferred embodiment of the present invention, a cellulosic material is used for the biodegradable bandage support member 13 due to the desirable material characteristics inherent in cellulosic materials such as low environmental impact and ease of biodegrading.

In the current embodiment of the present invention, the shape of biodegradable bandage support member 13 is formed from a square with an overlapping elongated rectangle. An embodiment of the present invention using biodegradable bandage support member 13 would allow for a decrease in surface area while not reducing the amount of active ingredients 19 applied to the biodegradable actives carrier 17. As described above in FIG. 1, other bandage support member 13 shapes can be selected from the group consisting of circular, rectangular, square, irregular and combinations thereof to suit individual applications.

The biodegradable adhesive 15 is located on a portion of the present invention biodegradable wound care product with biocompatible artificial skin treatment 11. This biodegradable adhesive 15 is adapted to secure the present invention biodegradable wound care product with biocompatible artificial skin treatment 11 to a user while still being biodegradable for disposal after use. The biodegradable adhesive 15 may be selected from the group consisting of biodegradable synthetic adhesives, biodegradable natural adhesives, and combinations thereof. In a preferred embodiment of the present invention, the biodegradable adhesive 15 includes a starch-based adhesive. Starch-based adhesives are readily available and biodegradable, making them an ideal adhesive for the present invention.

Biodegradable actives carrier 17 is attached to a portion of the biodegradable bandage support member 13. In the embodiment of the present invention shown in FIG. 3, the biodegradable actives carrier 17 may be constructed out of a material selected from the group consisting of biodegradable natural fiber textiles, biodegradable synthetic fiber textiles, biodegradable papers, biodegradable plastics and combinations thereof.

The biodegradable actives carrier 17 is adapted to hold an active ingredient 19 and release active ingredient 19 in a controlled manner. The active ingredient 19 is a paste derived from algae that has been dried, converted to a powder, and reconstituted with sterilized water. At least sixty percent by weight of the active ingredient 19 is reconstituted seaweed. Other actives or inerts may be added to the active ingredient 19 to achieve improved wound healing characteristics. In some preferred embodiments of the present invention biodegradable wound care product with biocompatible artificial skin treatment 11 the algae used is Laminaria seaweed. Laminaria seaweeds are desirable due to their high concentrations of poly-fucose sulfate, a fucodin that has been shown to accelerate wound-healing rates.

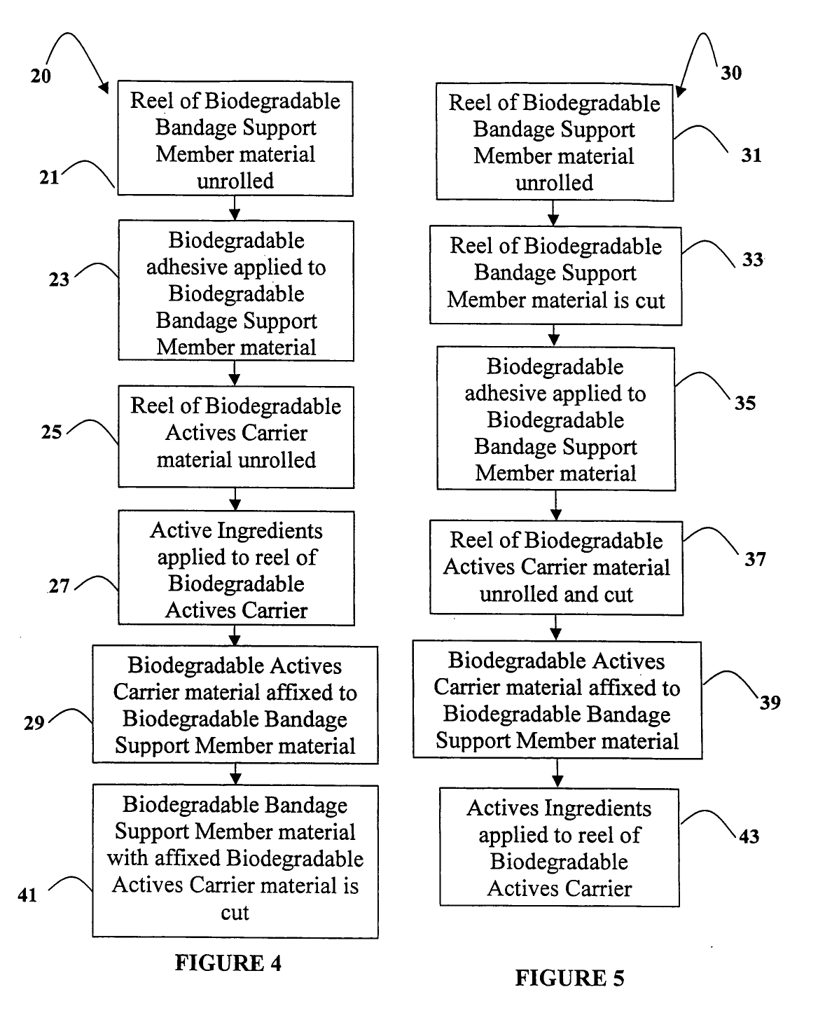

FIG. 4 is a flowchart 20 of one possible production method for the present invention biodegradable wound care product with biocompatible artificial skin treatment. Flowchart 20 outlines the basic steps 21, 23, 25, 27, 29, 41 of manufacturing the present invention biodegradable wound care product with biocompatible artificial skin. The first step 21 is to unroll the reel of biodegradable bandage support member raw material. As described above, a number of materials may be used, and reel is used here as an example raw material storage form and is not to be considered limiting. Raw material may be stored in reels, sheets or any other method commonly used. Once the biodegradable bandage support member has been unrolled, the second step 23 is to apply the biodegradable adhesive to the biodegradable bandage support member. One preferred method of applying the biodegradable adhesive would be through a spray applicator, although other application methods may be used. Step three 25 involves unrolling the reel of biodegradable actives carrier material. The active ingredients are applied to the biodegradable actives carrier material during step four 27. Step three 25 and step four 27 may be done after step two 23 or in tandem with step one 21 and step two 23. Once step four 27 is complete, the biodegradable actives carrier material is affixed to the biodegradable bandage support member. The final step 41 in the manufacturing process is to cut the biodegradable bandage support member with the affixed biodegradable actives carrier into individual units.

FIG. 5 is a flowchart 30 of another production method for the present invention biodegradable wound care product with biocompatible artificial skin treatment. The first step 31 is to unroll the reel of biodegradable bandage support member raw material. As described above, a number of materials may be used, and reel is used here as an example raw material storage form and is not to be considered limiting. Raw material may be stored in reels, sheets or any other method commonly used. Once the biodegradable bandage support member has been unrolled, the second step 33 is to cut the biodegradable bandage support member material into individual units. The biodegradable adhesive is then applied to the individual bandage support members in step three 35. Step four 37 involves unrolling the reel of biodegradable actives carrier material and cutting it into individual units. The active ingredients are then applied to the biodegradable actives carriers during step five 39. Step four 37 and step five 39 may be done after step two 33 or in tandem with step one 31 and step two 33. Once step five 39 is complete, the biodegradable actives carrier material is affixed to the biodegradable bandage support member in the final step 43.

Although FIG. 4 and FIG. 5 demonstrate two production methods for manufacturing the present invention biodegradable wound care product with biocompatible artificial skin treatment, it is to be understood that these two figures do not represent the only manufacturing methods. FIG. 4 and FIG. 5 serve to outline the individual steps necessary in the manufacturing process, although the order of the steps may be rearranged to suit manufacturing capabilities or design needs. For example, in FIG. 4 the second step 23 of applying the biodegradable adhesive may be done as a final step after the support member with affixed actives carrier is cut without changing the finished product.

The following three examples serve to demonstrate the preparation of three different embodiments of the present invention biodegradable wound care product with biocompatible artificial skin treatment.

### EXAMPLE 1

One kilogram or Laminaria seaweed is dried in an oven at 150° F. for six hours. The dried Laminaria seaweed is then ground to a fine powder with granule sizes around 0.5 mm. 400 grams of the dried, granulated Laminaria seaweed is then mixed with 200 grams of sterilized water and mixed to form a consistent paste with approximately 67% Laminaria seaweed by weight. The effect of this active is to promote healing of damaged tissue.

A biodegradable wound care product is prepared for use with the above active. A bio-polymer composed of starch and biodegradable polymers or polylactic acid is used as the biodegradable bandage support member. Bio-polymers are biodegradable and allow for breathing, which is beneficial in a wound care application. A simple biodegradable adhesive consisting primarily of starch is applied to one side the bandage support member. A mesh of woven cotton is used as a biodegradable actives carrier and attached to the center of the bandage support member on the same side as the biodegradable adhesive. Cotton is an organic material that is both biodegradable and absorbent, making it a good choice for the biodegradable actives carrier. The actives paste described in the above paragraph is applied to the biodegradable actives carrier and the entire assembly is cut into a single usable piece. This single piece is then sealed in a biodegradable wax paper to seal in the moisture and prevent the actives paste from drying out.

### EXAMPLE 2

One kilogram or Laminaria seaweed is dried in an oven at 150° F. for six hours. The dried Laminaria seaweed is then ground to a fine powder with granule sizes around 0.5 mm. 400 grams of the dried, granulated Laminaria seaweed is then mixed with 200 grams of sterilized water and 50 grams of Menthol and mixed to form a consistent paste with approximately 7.7% Menthol and 62% Laminaria seaweed by weight. This forms an actives compound that features topical pain reliever in addition to the active compound described in Example 1.

A biodegradable wound care product is prepared for use with the above active. A bio-polymer composed of starch and biodegradable polymers or polylactic acid is used as the biodegradable bandage support member. Bio-polymers are biodegradable and allow for breathing, which is beneficial in a wound care application. A simple biodegradable adhesive consisting primarily of starch is applied to one side the bandage support member. A mesh of woven cotton is used as a biodegradable actives carrier and attached to the center of the bandage support member on the same side as the biodegradable adhesive. Cotton is an organic material that is both biodegradable and absorbent, making it a good choice for the biodegradable actives carrier. The actives paste described in the above paragraph is applied to the biodegradable actives carrier and the entire assembly is cut into a single usable piece. This single piece is then sealed in a biodegradable wax paper to seal in the moisture and prevent the actives paste from drying out.

### EXAMPLE 3

One kilogram or Laminaria seaweed is dried in an oven at 150° F. for six hours. The dried Laminaria seaweed is then ground to a fine powder with granule sizes around 0.5 mm. 500 grams of the dried, granulated Laminaria seaweed is then mixed with 200 grams of sterilized water and 100 grams of a topical hemostatic powder and mixed to form a consistent paste with approximately 12.5% hemostatic powder and 63% Laminaria seaweed by weight. This forms an actives compound that features anticoagulation proprieties in addition to the active compound described in Example 1.

A biodegradable wound care product is prepared for use with the above active. A bio-polymer composed of starch and biodegradable polymers or polylactic acid is used as the biodegradable bandage support member. Bio-polymers are biodegradable and allow for breathing, which is beneficial in a wound care application. A simple biodegradable adhesive consisting primarily of starch is applied to one side the bandage support member. A mesh of woven cotton is used as a biodegradable actives carrier and attached to the center of the bandage support member on the same side as the biodegradable adhesive. Cotton is an organic material that is both biodegradable and absorbent, making it a good choice for the biodegradable actives carrier. The actives paste described in the above paragraph is applied to the biodegradable actives carrier and the entire assembly is cut into a single usable piece. This single piece is then sealed in a biodegradable wax paper to seal in the moisture and prevent the actives paste from drying out.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. A biodegradable wound care product with biocompatible artificial skin treatment, which comprises:
a) a biodegradable bandage support member and a biodegradable adhesive on at least a portion thereof, said support member being made of a biodegradable material;
b) a biodegradable actives carrier attached to a portion of said biodegradable bandage support member;
c) an active ingredient located on said biodegradable actives carrier, said active ingredient including a layer of biocompatible algae artificial skin including sterilized water and at least sixty percent by weight of reconstituted seaweed.

2. The biodegradable wound care product with biocompatible artificial skin treatment of claim 1 wherein said biodegradable bandage support member is a biodegradable bandage material selected from the group consisting of biodegradable natural fiber textiles, biodegradable synthetic fiber textiles, biodegradable papers, biodegradable plastics and combinations thereof.

3. The biodegradable wound care product with biocompatible artificial skin treatment of claim 1 wherein at least one of said biodegradable bandage support members is a cellulosic material.

4. The biodegradable wound care product with biocompatible artificial skin treatment of claim 1 wherein said biodegradable adhesive is selected from the group consisting of biodegradable synthetic adhesive, biodegradable natural adhesive, and combinations thereof.

5. The biodegradable wound care product with biocompatible artificial skin treatment of claim 4 wherein said biodegradable adhesive contains a starch-based adhesive.

6. The biodegradable wound care product with biocompatible artificial skin treatment of claim 1 wherein said active ingredient is a paste derived from algae that has been dried, converted to a powder, and reconstituted with sterilized water.

7. The biodegradable wound care product with biocompatible artificial skin treatment of claim 1 wherein said biodegradable actives carrier is selected from the group consisting of biodegradable natural fiber textiles, biodegradable synthetic fiber textiles, biodegradable papers, biodegradable plastics and combinations thereof.

8. The biodegradable wound care product with biocompatible artificial skin treatment of claim 1 wherein said bandage support member has a shape, said shape selected from the group consisting of circular, rectangular, square, irregular and combinations thereof.

9. The biodegradable wound care product with biocompatible artificial skin treatment of claim 1 further comprising a decorative design on said bandage support member.

10. The biodegradable wound care product with biocompatible artificial skin treatment of claim 1 wherein said bandage support member has a predetermined surface area, said surface area is determined by the desired application.

11. A biodegradable wound care product with biocompatible artificial skin treatment, which comprises:
a) a biodegradable bandage support member and a biodegradable adhesive on at least a portion thereof, said support member being made of a biodegradable material;
b) a biodegradable actives carrier attached to a portion of said bandage support member;
c) an active ingredient located on said biodegradable actives carrier, said biodegradable active ingredient including a layer of biocompatible algae artificial skin including sterilized water and at least sixty percent by weight of reconstituted Laminaria seaweed.

12. The biodegradable wound care product with biocompatible artificial skin treatment of claim 11 wherein said biodegradable bandage support member is a biodegradable bandage material selected from the group consisting of biodegradable natural fiber textiles, biodegradable synthetic fiber textiles, biodegradable papers, biodegradable plastics and combinations thereof.

13. The biodegradable wound care product with biocompatible artificial skin treatment of claim 11 wherein at least one of said biodegradable bandage support member is a cellulosic material.

14. The biodegradable wound care product with biocompatible artificial skin treatment of claim 11 wherein said biodegradable adhesive is selected from the group consisting of biodegradable synthetic adhesive, biodegradable natural adhesive, and combinations thereof.

15. The biodegradable wound care product with biocompatible artificial skin treatment of claim 14 wherein said biodegradable adhesive contains a starch-based adhesive.

16. The biodegradable wound care product with biocompatible artificial skin treatment of claim 11 wherein said active ingredient is a paste derived from algae that has been dried, converted to a powder, and reconstituted with sterilized water.

17. The biodegradable wound care product with biocompatible artificial skin treatment of claim 11 wherein said biodegradable actives carrier is selected from the group consisting of biodegradable natural fiber textiles, biodegradable synthetic fiber textiles, biodegradable papers, biodegradable plastics and combinations thereof.

18. The biodegradable wound care product with biocompatible artificial skin treatment of claim 11 wherein said bandage support member has a shape, said shape selected from the group consisting of circular, rectangular, square, irregular and combinations thereof.

19. The biodegradable wound care product with biocompatible artificial skin treatment of claim 11 further comprising a decorative design on said bandage support member.

20. The biodegradable wound care product with biocompatible artificial skin treatment of claim 11 wherein said bandage support member has a predetermined surface area, said area is determined by the desired application.
